(19) United States
(12) Patent Application Publication
(10) Pub. No.: US 2019/0015614 A1
(43) Pub. Date: Jan. 17, 2019

(54) SYSTEMS, DEVICES, AND METHODOLOGIES TO PROVIDE PROTECTIVE AND PERSONALIZED VENTILATION

(71) Applicant: ROYAL COMMISSION YANBU COLLEGES & INSTITUTES, Yanbu al-Sinaiyah (SA)

(72) Inventor: Husam Ibrahim ALAHMADI, Yanbu al-Sinaiyah (SA)

(73) Assignee: ROYAL COMMISSION YANBU COLLEGES & INSTITUTES, Yanbu al-Sinaiyah (SA)

(21) Appl. No.: 15/648,949

(22) Filed: Jul. 13, 2017

(51) Int. Cl. A61M 16/00; A61M 16/10; A61M 16/12; A61M 16/16; A61M 16/20

(52) U.S. Cl. CPC A61M 16/0051; A61M 16/0069; A61M 16/104; G06F 19/3431; A61M 16/161; A61M 16/202; A61M 16/12

(57) ABSTRACT

A method and system for monitoring respiratory waveforms. The method includes acquiring a data set representative of a waveform, comparing one or more segments of the data set with stored abnormal shapes and/or values, determining, using the processing circuitry and based on the comparison, a match level, identifying an abnormality associated with an abnormal shape and/or a value in response to determining that the match level between the data set and the abnormal shape and/or the value is above greater or below a predetermined threshold, and outputting a notification indicating the abnormality to an external device.

### BACKGROUND

Mechanical ventilation (MV) is used to mechanically assist or replace spontaneous breathing using a mechanical ventilator. The mechanical ventilator is applied whenever there is a clinical indication. Patients with high work of breathing, inadequate minute ventilation and apnea are some examples. The ventilator may be operated in multiple modes, parameters based on the patient's neurological and mechanical abilities, medical history, nature of insult/disease, and clinical statues/goals.

There are certain risks associated with mechanical ventilation. The mechanical ventilation may cause lung injury as a result of stress and/or strain. Excessive pressures and/or tidal volume for a given patient can lead to ventilator-induced lung injury (VILI). Another common risk is asynchrony between a patient and the mechanical ventilator. Failure to detect and treat patient ventilator asynchrony may lead to untoward complications. Such as increased patient agitation, sedation, prolonged time on the mechanical ventilator, intensive care unit (ICU) stay thus increasing the risk of hospital-acquired infection, mortality, and cost of care.

The foregoing “Background” description is for the purpose of generally presenting the context of the disclosure. Work of the inventor, to the extent it is described in this background section, as well as aspects of the description which may not otherwise qualify as prior art at the time of filing, are neither expressly or impliedly admitted as prior art against the present invention.

### SUMMARY

The present disclosure relates to a method for monitoring respiratory waveforms. The method includes acquiring a data set representative of a waveform, comparing one or more segments of the data set with stored abnormal shapes and/or values, determining, using the processing circuitry and based on the comparison, a match level, identifying as abnormality associated with an abnormal shape and/or a value in response to determining that the match level between the data set and the abnormal shape and/or the value is above greater or below a predetermined threshold, and outputting a notification indicating the abnormality to an external device.

In another aspect, the present disclosure relates to a mechanical ventilator system. The mechanical ventilator system includes a mechanical ventilator and processing circuitry. The processing circuitry is configured to acquire a data set representative of a waveform from the mechanical ventilator, compare one or more segments of the data set with stored abnormal shapes and/or values, determine a match level based on the comparison, identify an abnormality associated with an abnormal shape and/or a value in response to determining that the match level between the data set and the abnormal shape and/or value is above greater or below a predetermined threshold, and output a notification indicating the abnormality to an external device.

The foregoing paragraphs have been provided by way of general introduction, and are not intended to limit the scope of the following claims. The described embodiments, together with further advantages will be best understood by reference to the follow my detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a schematic diagram that shows an example environment for an abnormality detection system according to one example;

FIG. 2 is an exemplary diagram of waveforms data;

FIG. 3 is a flowchart of an abnormalities detection process according to one example;

FIG. 4 is a flowchart of a monitoring process according to one example;

FIG. 5 is a flowchart of a leak detection process according to one example;

FIGS. 6A-6L are schematics that show exemplary predetermined waveforms associated with abnormalities;

FIG. 7 is an exemplary block diagram of a computer recording to one example;

FIG. 8 is an exemplary block diagram of a data processing system according to one example; and

FIG. 9 is an exemplary block diagram of a central processing unit according to one example.

### DETAILED DESCRIPTION

Referring now to the drawings, wherein like reference numerals designate identical or corresponding parts throughout several views, the following description relates to a system and associated methodology for monitoring and detecting abnormalities in pulmonary mechanics. The system described herein provides a personalized and customized monitoring to enhance patient safety when using a mechanical ventilator by preventing or minimizing lung injuries/infections and optimizing patient-ventilator synchrony.

The mechanical ventilator is a breathing machine used to assist and/or replace the spontaneous breathing of critically ill patients. The mechanical ventilator can be applied invasively or non-invasively. The methodologies described herein may be applied to waveforms acquired from mechanical ventilators irrespective of the ventilator mode, interface of operation (i.e., invasively or non-invasively). A mechanical breath may be initiated by the patient (i.e., patient trigger) or as a function of time (i.e., time trigger).

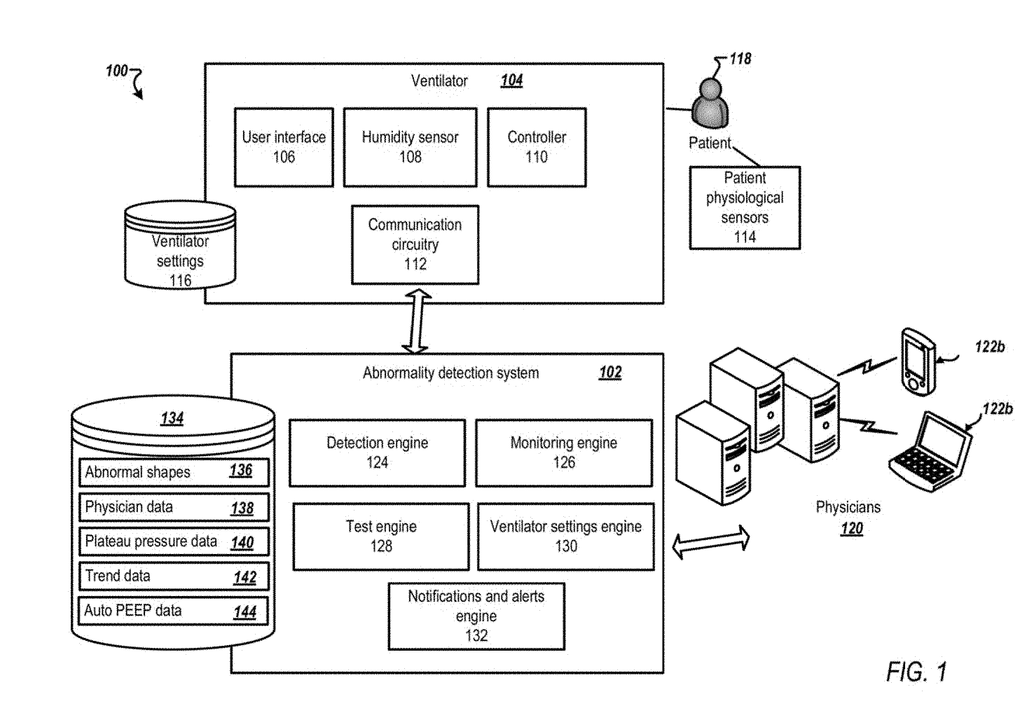

FIG. 1 is a schematic diagram of an example environment 100 for an abnormality detection system 102 according to one example. The abnormality detection system 102 is configured to detect abnormalities in respiratory waveforms ( i.e., graphics, pulmonary mechanics). For example, the abnormality detection system 102 can detect abnormalities in waveforms acquired from a mechanical ventilator 104. Further, the abnormality detection system 102 may detect air leaks during mechanical ventilation. In addition, the abnormality detection system 102 automatically performs and analyzes routine monitoring maneuvers as described further below.

The mechanical ventilator 104 has a user interface 106, for example, a touch sensitive display, via which a physician, a respiratory specialist, or other medical personnel can enter or adjust ventilator settings 116. The mechanical ventilator 104 may also include a humidity sensor 108, a controller 110, and communication circuitry 112. The mechanical ventilator 104 may also acquire data from patient physiological sensors 114. The mechanical ventilator 104 delivers air flow in accordance with the ventilator settings 116 to a ventilated patient 118.

The patient physiological sensors 114 may include a flowmeter measuring airway flow rate, a pressure gauge measuring airway pressure, and a capnography measuring carbon dioxide in respiration gases. In addition, the patient physiological sensors 114 may include sensors that monitor heart rate, blood pressure (e.g., arterial blood pressure, central venous pressure), and oxygen saturation (e.g., SpO₂ level).

The humidity sensor 108 can measure relative and/or absolute humidity level of inspired air delivered to the ventilated patient 118. Adequate humidification of the inspired air is vital and mandatory when invasive (i.e., upper airway is bypassed) ventilation is applied. The abnormality detection system 102 can check to see whether the humidity level is within a predetermined humidification range.

The abnormality detection system 102 can receive information from the mechanical ventilator 104. The information may be provided in the form of one or more waveforms and/or one or more datasets that may be used to construct a waveform.

In one implementation, five ventilator waveforms may be monitored. Three scalars which monitors the pressure (P), the volume (V), and the flow (F) with respect to time. In addition, two loop waveforms may be monitored. The loop waveforms represent scalar values with respect to each other. For example, a first loop waveform may represent flow versus volume known as F-V loop. Flow is plotted on the y axis and volume is plotted on the x axis. A second loop waveform may be pressure versus volume (PV loop). Additional waveforms may also be monitored based on a ventilator model or a request from physician 120. The additional waveforms may include capnography, electrical activity of the diaphragm (Edi), esophageal pressure (Peo), transpulmonary pressure (Ptp), SpO₂, electroencephalogram (EEG), electromyography (EMG), electrooculography (EOG), nasal pressure graph, thermistor graph, and humidity graph.

The measurement and display of waveforms takes place during the inspiration and expiration phases of a respiratory cycle. Inspiratory/expiratory flow may be used to identify airway obstruction. On the other hand volume may be used to identify volume restriction (leak).

The pressure scalar may be based on data collected via a pressure transducer. The flow scalar may be based data collected via pneumotochographs, fixed and/or variable orifice meters, hot wire anemometers, ultrasonic flowmeters, and the like. The volume scalar may be obtained using an electronic integrator that estimate volume by passing flow signals. The pressure volume loop can be based via data from a pressure transducer or a flow sensor. The flow volume loop may be based on data collected via the flow sensor or the electronic integrator.

The capnography is based on data collected via the CO₂ sensor. The Edi waveform is collected via data obtained from electrodes that transmits signal to the mechanical ventilator 104. The Esophageal pressure is obtained via the pressure transducer. The SpO₂ graph may be obtained from data obtained via the SpO₂ sensor.

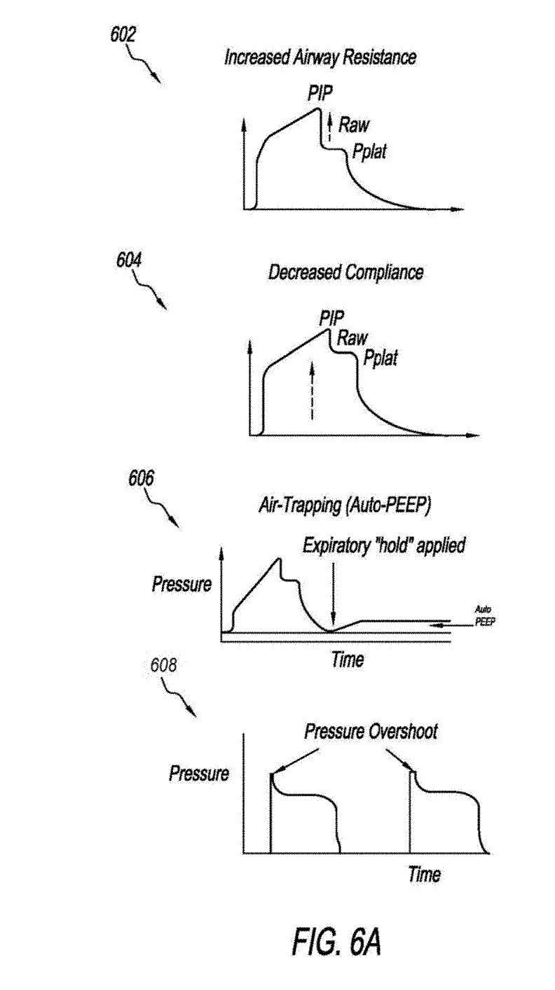

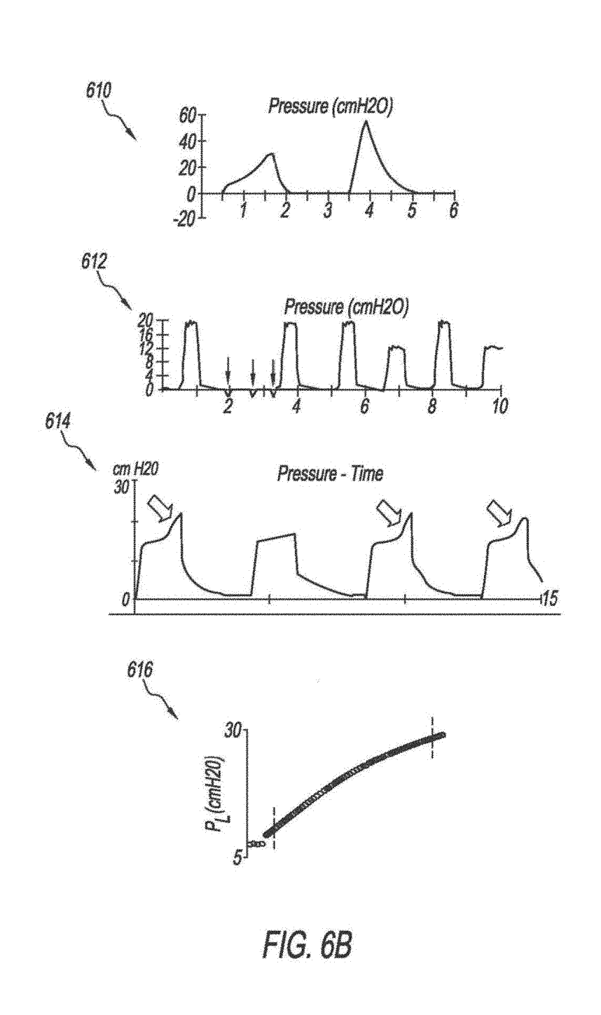

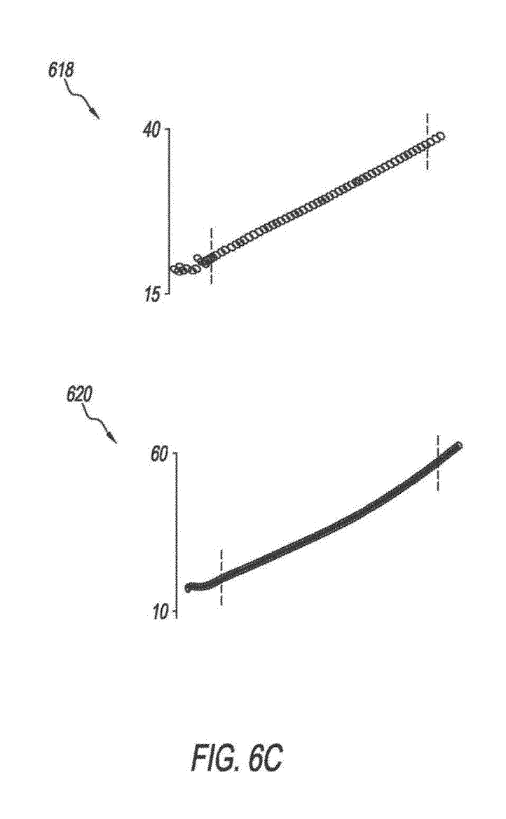

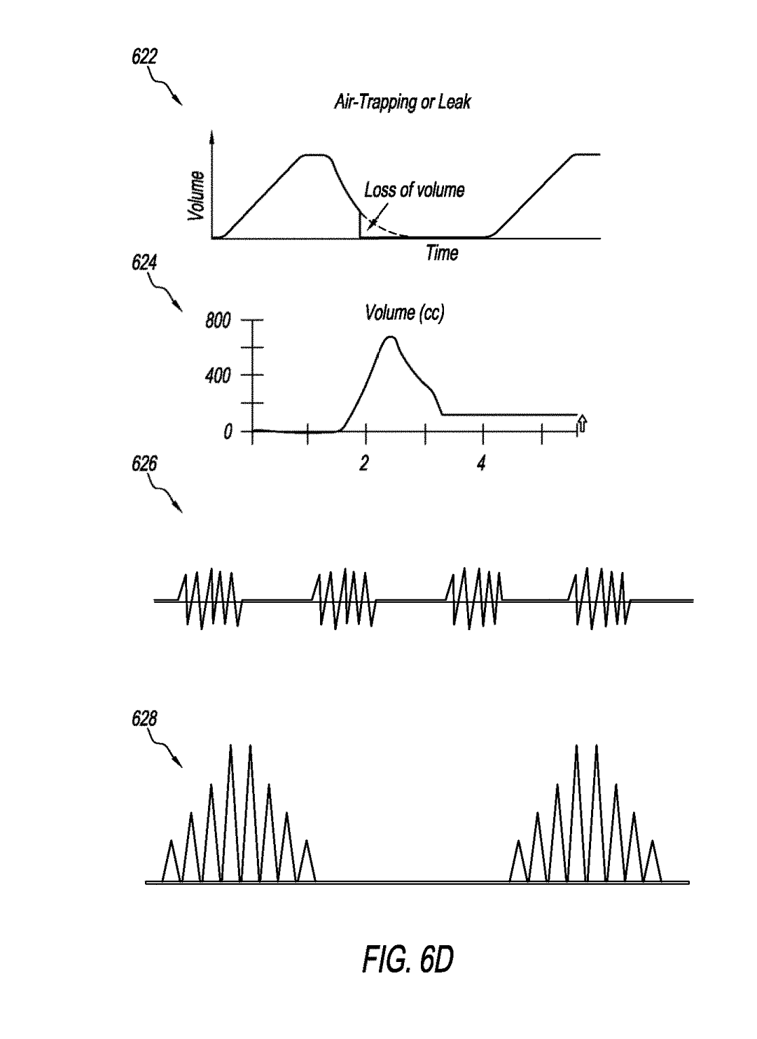

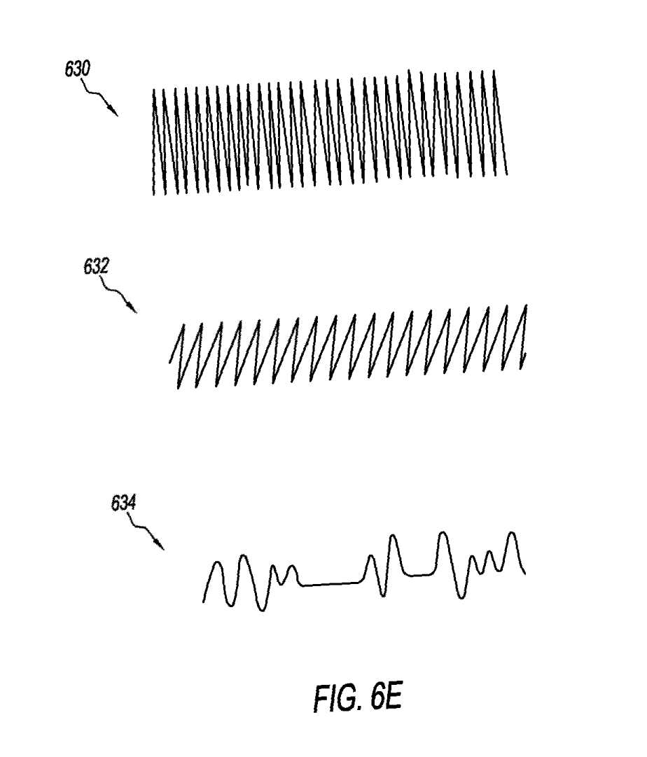

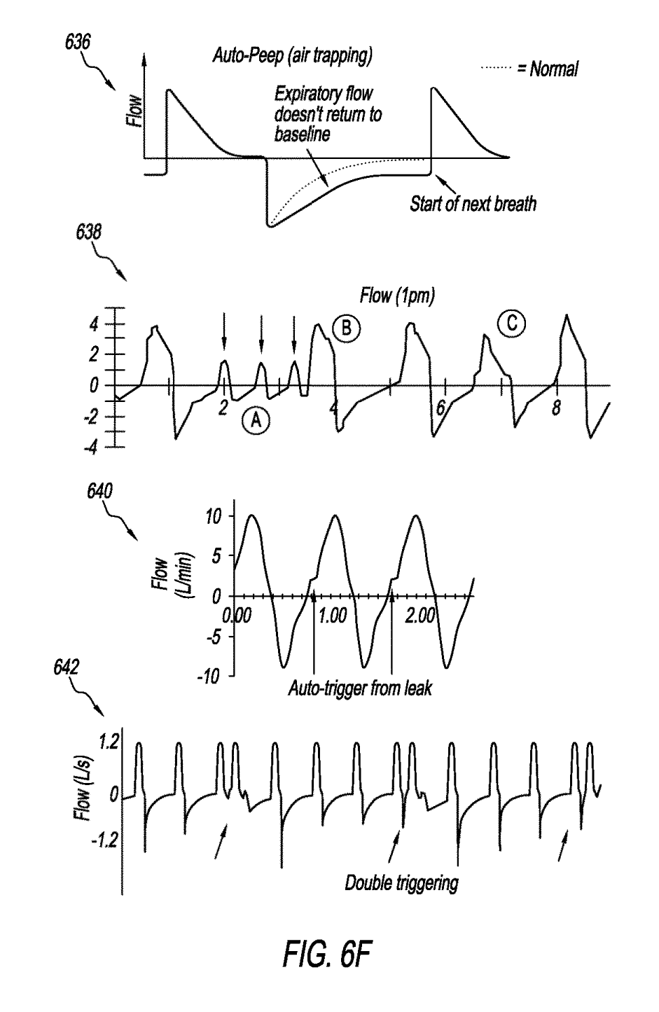

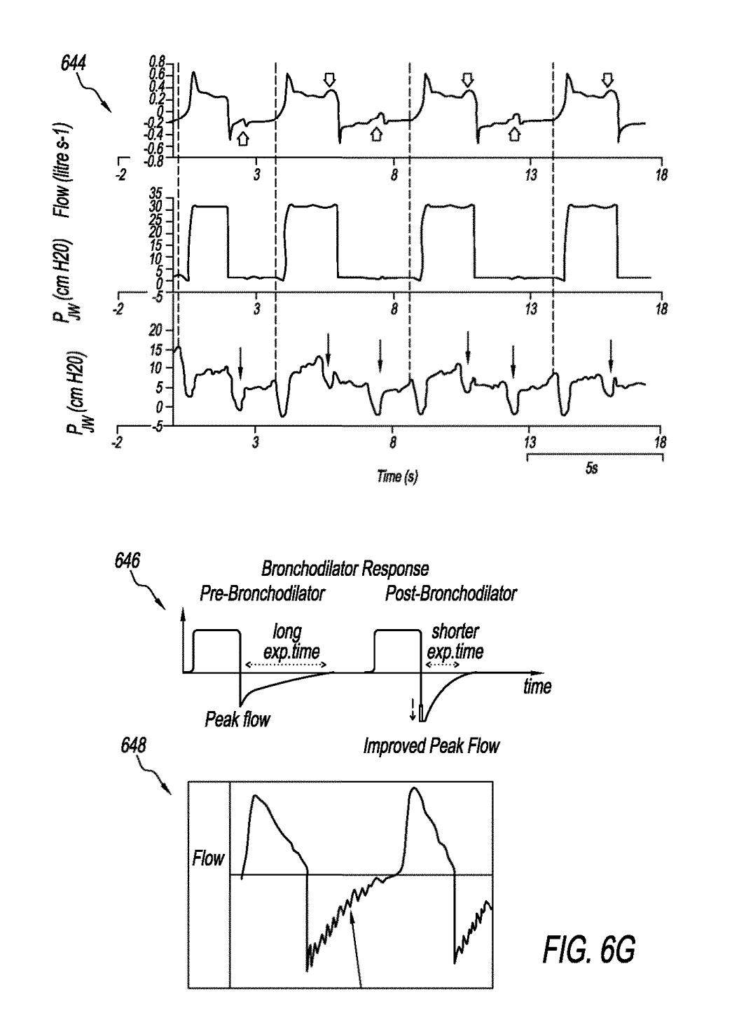

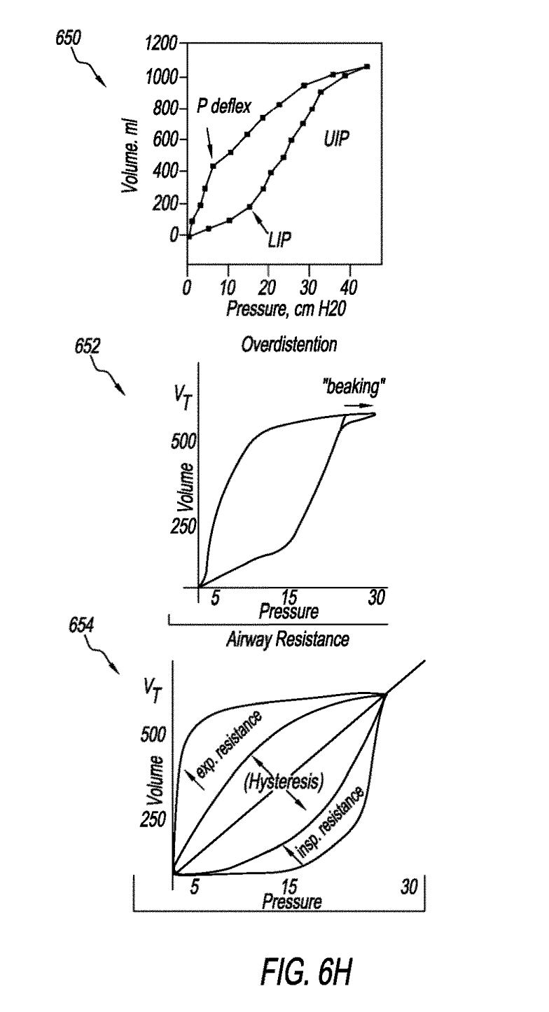

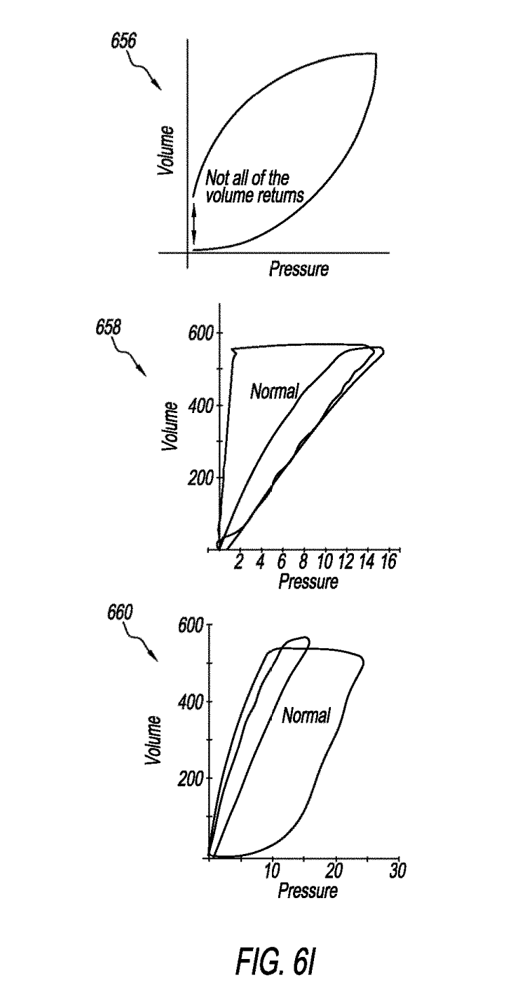

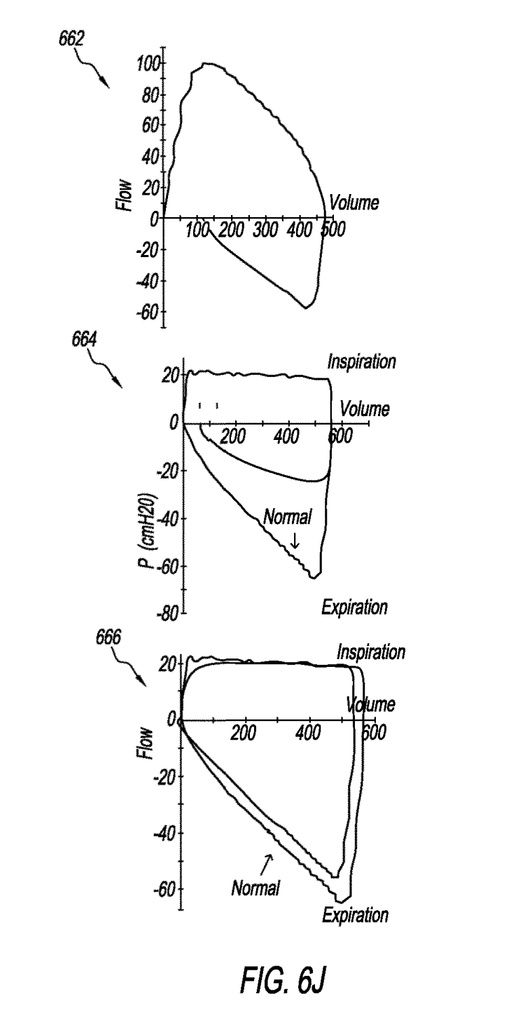

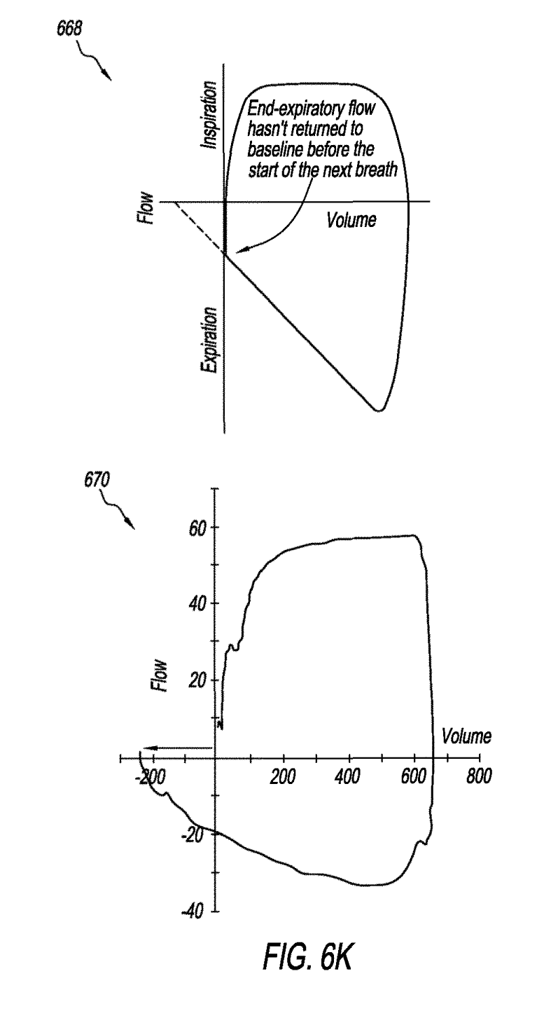

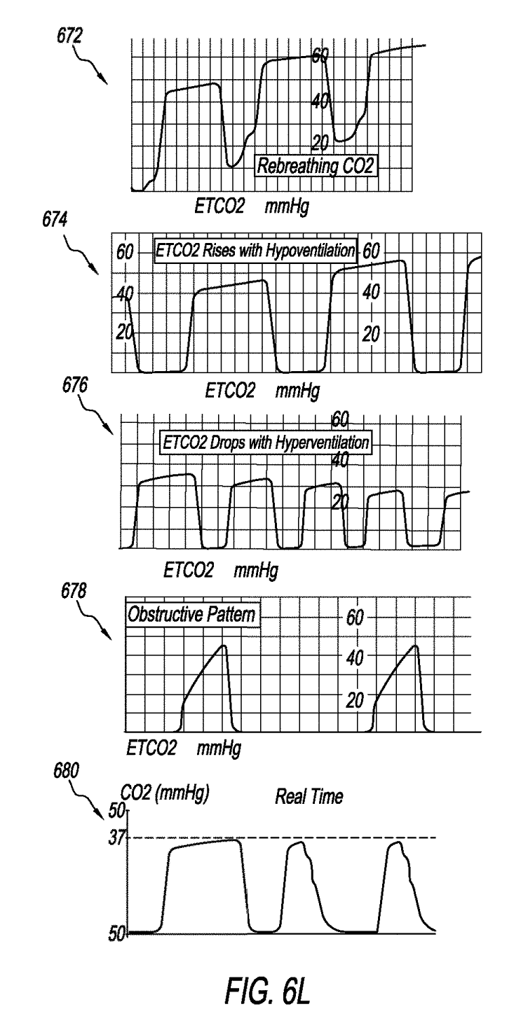

Using advanced interpretation of the ventilator waveforms, the system described herein provides protective mechanical ventilation to patients. Abnormalities are associated with specific shapes and/or values identified in the ventilator waveforms. Exemplary abnormal shapes are shown in FIGS. 6A-6L.

Physicians 120 can include medical doctors, respiratory specialists, clinicians, caregivers, or any other authorized medical personnel who are monitoring one or more patients via one or more computing devices 122 that include mobile device 122a, computer 122b, or any other type of external computing device. The physicians 120 can access the abnormality detection system 102 to track a patient 118. Further, the physicians 120 may approve/disapprove ventilation settings changes determined by the abnormality detection system 102 as described further below.

The mechanical ventilator 104 and physicians 120 can connect to the abnormality detection system 102 via a wired or wireless network (not shown). The network can include one or more networks, such as the Internet and can also communicate via wireless networks such as WI-FI, BLUETOOTH, cellular networks including EDGE, 3G, and 4G wireless cellular systems, or any other wireless form of communication that is known that is pre-registered, verified, and highly secured.

The abnormality detection system 102 includes one or more engines or modules that perform process associated with receiving ventilator waveforms from one or more mechanical ventilators 104, analyzing the ventilator waveforms to identify one or more abnormalities, activating one or more monitoring tests, monitoring various sensors to identify existing or potential problems, and alerting the physicians 120 when an abnormality or potential abnormality is detected. Further, the abnormality detection system 102 may determine updated ventilator settings based on the abnormal sties detected.

References to the engines or modules throughout the disclosure are meant to refer to software and/or hardware processes executed by circuitry of one or more processing circuits, which can also be referred to interchangeably as processing circuitry. In some implementations, the processes associated with the abnormality detection system 102 can be performed by one or more servers having one or more processing circuit such that some steps may be performed on different servers.

The modules described herein may be implemented as either software and/or hardware modules and may be stored in any type of computer-readable medium or other computer storage device. For example, each of the modules described herein may be implemented in circuitry that is programmable (e.g. microprocessor-based circuits) or dedicated circuits such as application specific integrated circuits (ASICS) or field programmable gate arrays (FPGAS). In one embodiment, a central processing unit (CPU) could execute software to perform the functions attributable to each of the modules described herein. The CPU may execute software instructions written in a programing language such as Java, C, or assembly. One or more software instructions in the modules may be embedded in firmware, such as an erasable programmable read-only memory (EPROM).

In one example, the abnormality detection system 102 includes a detection engine 124 that detects abnormalities in the ventilator waveforms received from the mechanical ventilator 104. In one implementation, the detection engine 124 may identify abnormalities based on data stored in data repository 134 as abnormal shapes data 136, which can be a database of data files of predetermined shapes associated with abnormal conditions (abnormalities).

The abnormal shapes data 136 may include shapes associated with a complete respiratory cycle or associated with a segment of the respiratory cycle as described further below. In addition, each shape may be associated with one or more abnormalities and with one or more waveforms. The abnormal shapes data 136 may also include derivatives of the abnormal shapes (e.g., first derivative), normalized shapes, scaled shapes, and the like.

The abnormality detection system 102 may include a monitoring engine 126 that monitor a patient status and monitor leaks in the mechanical ventilator 104. The monitoring engine 126 receives as inputs ventilator output information including ventilator waveforms. The monitoring engine 126 may also acquire physiological variables that may be monitored by the patient physiological sensors 114.

The monitoring engine 126 is further configured to track waveform data of the patient 118 tor a predetermined period (e.g., last 24 hours or 48 hours) to determine a status of the patient 118. For example, the monitoring engine 126 may classify the status of the patient 118 into “unchanged”, “improving”, or “deteriorating” based on the collected waveforms using artificial intelligence (e.g., classifier based on genetic algorithms). For example, the monitoring engine 126 may classify the patient based on a number of abnormalities detected within the predetermined period.

The abnormality detection system 102 may include a notifications and alerts engine 132. The notifications and alerts engine 132 can provide alerts to physicians 120 upon identifying an abnormality by the detection engine 124. Further, the notifications and alerts engine 132 may identify a particular physician associated with the mechanical ventilator 104 among the one or more physicians 120 by referencing a database, for example, physician data 138 stored in data repository 134. For example, one physician may be associated with multiple mechanical ventilators. Further, the physician data 138 maintains an up to date association based on physicians 120 inputs or other parties input. For example, the notifications and alerts engines 132 may retrieve information from an electronic calendar associated with the physician to determine whether the physician is on a break, or unavailable. The notifications and alerts engine 132 can also issue an alert to the physician 120 when the humidity level acquired from the humidity sensor 108 of the mechanical ventilator 104 is outside the predetermined range. Further, the notifications and alerts engine 132 may output reports that provide an objective assessment of patients' response to any therapeutic interventions such as bronchodilation therapy, airway clearance therapy, and the like.

The abnormality detection system 102 may include a ventilator settings engine 130. The ventilator settings engine 130 may determine updated settings and potential solutions based on the detected abnormalities. The ventilator settings engine 130 may communicate the updated settings to the physicians 120 via the notifications and alerts engine 132.

The mechanical ventilator 104 may include a close-loop operating mode. In a closed-loop operating mode, the pre-authorized settings adjustment is automatically applied without intervention of medical personnel. This approach advantageously enables very rapid (essentially real-time) response to a sudden change in the condition of the ventilated patient.

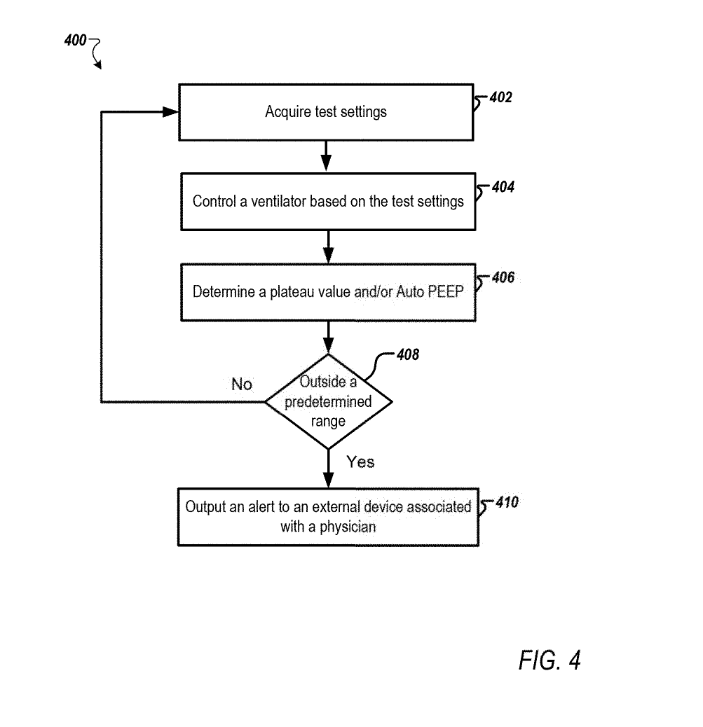

The abnormality detection system 102 may include a test engine 128. The test engine 128 may trigger medical checks at preset intervals that may include controlling the ventilator settings 116 and analyzing outputs from the mechanical ventilator 104 as described further below. An exemplary process to monitor a plateau pressure is shown and described in FIG. 4. The plateau pressure and thresholds associated with the plateau pressure may be stored as plateau pressure data 140 in the data repository 134.

In one implementation, the abnormality detection system 102 acquires a measure of exhaled nitric oxide (biological marker) to monitor the progression or regression of asthma exacerbation during invasive and/or non-invasive mechanical ventilation.

In one implementation, a certain level of pressure in Endotracheal tube cuff is continuously maintained via connecting a pilot balloon to a monometer inside the ventilator 104. Maintenance of appropriate (i.e., not over/under inflation) intra-cuff pressure is a critical factor to prevent or minimize aspiration (which is a major cause of ventilator-associated pneumonia (VAP) around the cuff due to low cuff pressure as well as to prevent or minimize tracheal hypoperfusion injury due to high cuff pressure (which cause airway edema that could cause extubation failure that may necessitate reintubation which increases the risk of VAP).

In one implementation, the abnormality detection system 102 may determine the apnea index that occurs during invasive mechanical ventilation that could be undetected. Apnea (i.e., absence of breathing) that occurs for longer than 10 seconds is considered abnormal and may be counted as 1 apnic episode. Apnea index is the number of apnic episodes per hour. Apnea that lasts less than 20 seconds may not detected because the common range for apnea time alarm setting is 20 to 30 seconds in adults. Thus, apnea that occurs between 10 to 20 seconds may not be detected. Apnea during invasive MV can be caused by central apnea, chemical (low CO₂) hyperventilation, high PS level (produces high tidal volume)/high trigger sensitivity setting, reflex (i.e., lung hyperinflation), ineffective triggering due to dynamic hyperinflation (intrinsic positive end-expiratory pressure), and/or low trigger sensitivity setting.

Although the description herein relates to ventilator waveforms, it is to be understood that the system described herein and associated methodologies may be applied to other waveforms and or values such as arterial line, central line, venous and central venous oxygen saturation, intracranial pressure, intra-aortic balloon pressure and Pulse Contour Cardiac Output.

In one implementation, the methodologies described herein may be implemented in pulmonary function tests (PFTs). For example, waveforms acquired during various PFTs may be input to the abnormality detection system 102. The abnormality detection system 102 may analyze the waveforms and/or the values using the methodologies described herein to detect any abnormality.

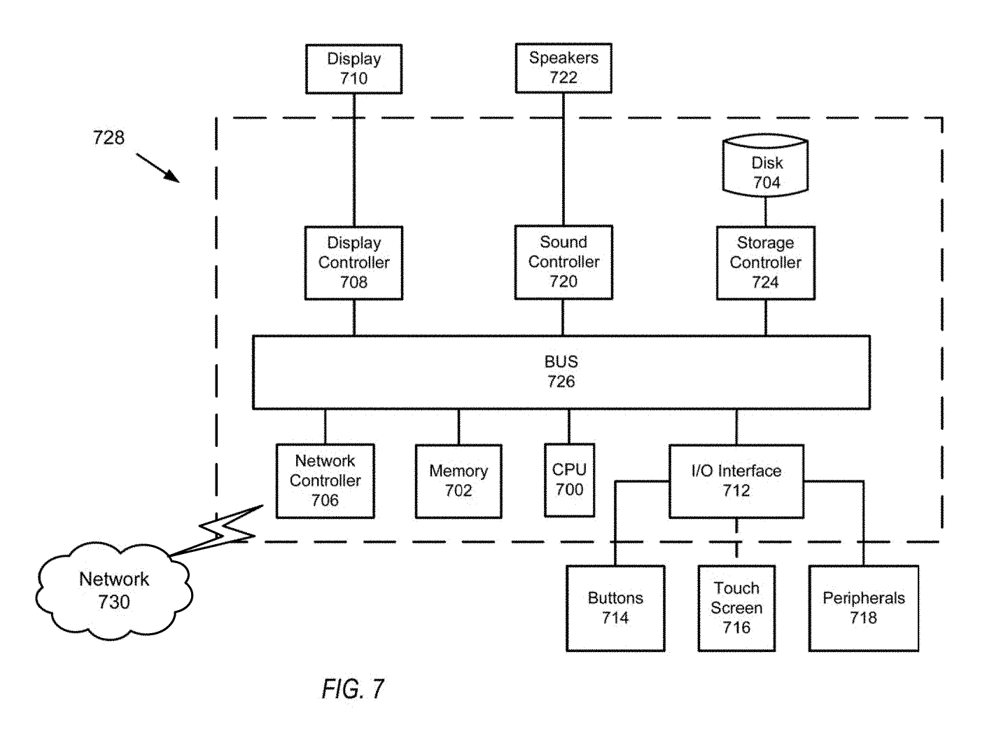

The description herein is provided with reference to the abnormality detection system 102 being located and implemented external to the mechanical ventilator 104. However, it is to be understood that the system may alternatively or additionally be implemented within the mechanical ventilator 104, where the mechanical ventilator 104 may contain hardware similar to that illustrated in FIG. 7, and the databases (e.g., data repository 134) of the system may correspond to a memory of the mechanical ventilator 104.

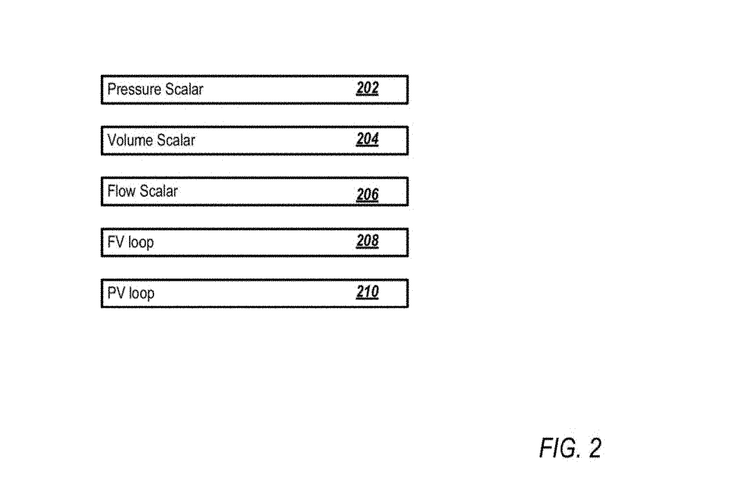

FIG. 2 is a schematic diagram of abnormal shape data 136 according to one example. The abnormal shapes data 136 may include shape data associated pressure scalar 202, volume scalar 204, flow scalar 206, FV loop 208, and PV loop 210 such as lower inflection point (LIP) and upper inflection point (UIP). Ventilating patients between LIP and UIP can achieve “protective lung strategy” a strategy used to prevent/minimize ventilator-induced lung injury (VILI).

Appearance of LDP and LIP indicates a probability of developing atelectotrauma (a type of VILI). For example, the appearance of an LDP in the PV loop 210 may indicate the beginning of lung collapse (i.e., derecruitment). Thus, the LDP is monitored to determine whether it is changing overtime. The abnormalities detected from the waveforms may include, but are not limited to, beak sign, air trapping intrinsic positive end-expiratory pressure (PEEP), flow starvation, active exhalation, premature inhalation, missed triggers, secretion/condensation accumulation, system leak, under or over humidification and abnormal breathing patterns.

A patient with lung problems may have a high amount of secretion (e.g., tracheal secretion, lower respiratory tract secretion). A common parameter to monitor airway resistance is peak inspiratory pressure (PIP). A less common but a more sensitive parameter is the pressure difference (delta) between PIP and Plateau pressure (ΔP_{PIP-Pplat}). The pressure difference between PIP and Pplat represents the airway resistive pressure. The upper pressure alarm limit is not always set appropriately or left at the default alarm setting which is too high for a relatively healthy patient. The default upper pressure alarm limit value (e.g., 40 cmH₂O) is reached when for example a large amount of secretion is present which may be an advanced stage. At this advanced stage, patients are usually agitated and associated with oxygen desaturation, which may risk patients for mucous plugs that could lead to atelectasis, auto PEEP, and hypoxic complications. Automated measuring, monitoring, and trending of the ΔP_{PIP-Pplat} serve as a brand-new assessment tool as described herein. There are several patient safety clinical benefits associated with integrating and implementing ΔP_{PIP-Pplat}: 1) ΔP_{PIP-Pplat} is a more sensitive indicator than PIP for airway resistance, and 2) ΔP_{PIP-Pplat} serves as an independent additional alarm setting regardless of the upper pressure alarm setting value. Alternatively, the predetermined shapes that are indicative of high airway resistance (e.g., the presence of secretion, bronchoconstriction) in the lungs allow an early detection/notification of the secretion accumulation or bronchoconstriction and prevents/minimizes the incidence of oxygen desaturation.

The ventilator settings 116 are patient dependent and optimal ventilator settings may continuously vary. For example, low values of positive end-expiratory pressure may cause alveoli units to collapse and hence result in poorly ventilated lungs. On the other hand, high values of PEEP may open up more alveoli units but may impair venous return hence result in low cardiac output (CO) and mean arterial blood pressure (MAP). Similarly, high values of FiO₂ may increase arterial blood oxygen partial pressure (PaO₂) but may have toxicity side effects. Too low values of tidal volume may result in inadequate ventilation, whereas too high tidal volume values may cause pulmonary volutrauma and barotrauma, depending on the mechanical properties of the patient's lungs. Further, the optimal value of respiratory rate (RR) to guarantee adetpate ventilation may depend upon the selected tidal volume. Pulmonary volutrauma is a microscopic injury affecting alveolar and pulmonary capillary walls. Volutrauma is caused by overstretching/overdistending of alveoli by the effect of excessive levels of tidal volume and/or inspiratory pressure. Volutrauma triggers an inflammatory cascade that may causes further lung injury.

Optimal PEEP is the level of PEEP that achieves PEEP clinical benefits. Such as highest (oxygen delivery, Functional Residual Capacity, and lung static compliance) with lowest pulmonary shunt ratio. Also, optimal PEEP is not associated with cardiovascular side effects. The optimal PEEP is patient dependent. The optimal PEEP has a characteristic shape that may be hard to identify at the clinical bedside. Pressure-Volume (P-V) loop can facilitate identifying the optimal PEEP level via analyzing P-V loop morphology. Optimal PEEP is defined as the level of PEEP that prevents the major parts of the lungs from collapse (de-recruitment). Another characteristic of PV loop is hysteresis (volume difference between inspiratory and expiratory on PV loop). It has been used to assess the level of lung recruitability that is associated with maximum hysteresis. Optimal PEEP level should be set 2-3 cmH₂O above the LIP. LIP means a significant increase in tidal volume (start of lung inflation). The LIP approximately takes place in the first quarter of the inhalation. Thus, the abnormality detection system 102 may analyze the LIP (continuously, frequently or on-demand) to determine whether the optimal PEEP is applied at all times to maximize the prevention of the atelectrauma incidence. The optimal PEEP level may vary even within the patient him/herself from time to time.

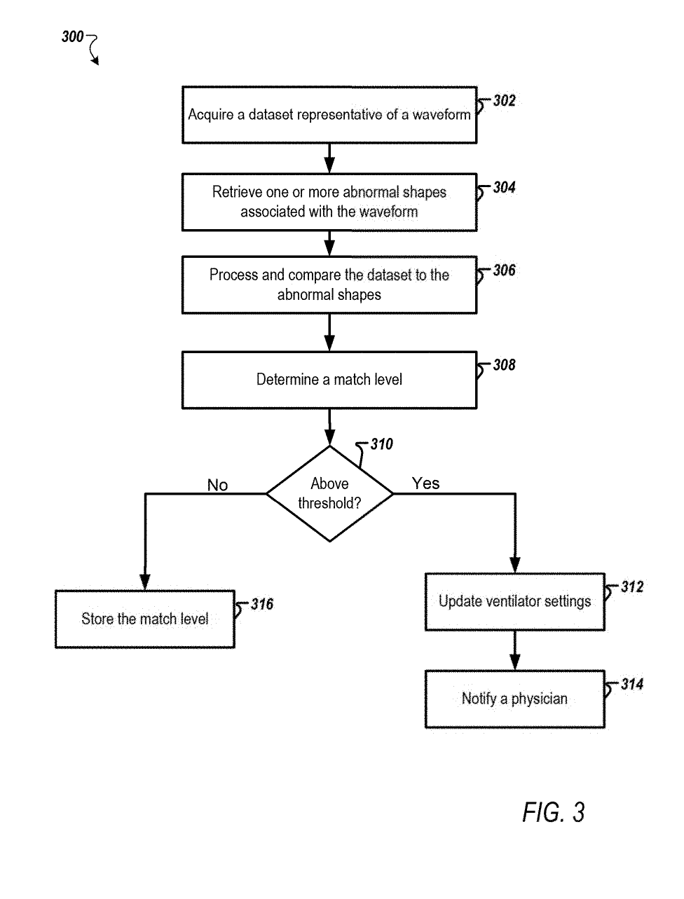

FIG. 3 is a flowchart of an abnormality detection process 300 according to one example. The abnormalities detection process 300 is performed by one or more of the processing engines of the abnormality detection system 102, such as the detection engine 124, the monitoring engine 126, and the test engine 128.

At step 302, the detection engine 124 acquires a data set representative of at least one waveform in real-time. The waveform may include one or more respiratory cycles. The detection engine 124 may also acquire multiple datasets corresponding to one or more waveforms data shown in FIG. 2.

At step 304, the detection engine 124 may determine a category of the waveform (e.g., pressure scalar 202, volume scalar 204, flow scalar 206, FV loop 208, PV loop 210). The category may be determined based on data acquired with the waveform data. In other implementations, the category is input by the user or predetermined. The detection engine 124 may retrieve one or more abnormal shapes associated with the category. As described previously herein, the one or more abnormal shapes are associated with abnormalities.

At step 306, the detection engine 128 may compare the acquired waveform with the one or more predetermined shapes using imaging analysis techniques. The detection engine 128 may also use pattern recognition techniques such as classification algorithms (e.g., neural networks, gene expression programming, naive Bayes classifier, genetic algorithm, simulated annealing), clustering algorithms (e.g., deep learning methods, correlation clustering), ensemble learning algorithms (e.g., ensemble averaging, bootstrap aggregating), and the like. Further, the detection engine 128 may determine a match level which is indicative of a level of closeness between the dataset and one abnormal shape associated with an abnormality, at step 308.

In one implementation, the dataset may be segmented to multiple segments which may be in function of the respiratory cycle (e.g., start, middle, end of the respiratory cycle). In other words, a portion of the waveform is compared to the abnormal shapes. Each segment is compared with predefined abnormal shapes associated with the corresponding segments. For example, a particular abnormal shape may appear at a particular location in the waveform corresponding to a specific phase of the respiratory cycle. By analyzing only a portion of the waveform and comparing the portion to relative predefined abnormal shapes, processing time is reduced. For example, pulmonary volutrauma take place at the end of inspiration phase of the respiratory cycle. During the inhalation phase of the respiratory cycle the lungs can start picking up at a normal capacity but that at the end of the inhalation the lungs are filled and the lungs can start to be overinflated and/or to be over distended. Thus, abnormality shapes associated with pulmonary volutrauma appears at the portion of the waveform associated with the end of inhalation phase of the respiratory cycle.

The detection engine 124 may determine a slope associated with predefined segments of the waveforms (e.g., each waveform may be divided into hundreds of segments). The slopes may be categorized into ascending, descending, or flat. Then, the slopes are compared with pre-stored slopes of abnormal shapes. By analyzing a slope of each segment preprocessing of the waveform may not be necessary and processing time is reduced.

In one example, a first derivative of the waveform or a segment of waveform may be taken. Then, the first derivative is compared with first derivatives of the abnormal shapes stored in abnormal shapes 136.

In one example, a frequency spectral comparison may be performed between the waveform or segments of the waveform and spectral representations of the abnormal shapes. For example, a digital Fourier transform (DFT) or wavelet of the waveform may be calculated and then compared with DFT representations or wavelet transforms of the abnormal shapes. The DFT representations of the abnormal shapes may be pre-calculated and stored in the abnormal shapes data 136. In one example, a short-time Fourier transform (STFT) of the waveform may be determined and compared with STFTs of abnormal shapes. The STFT determines sinusoidal frequency and phase content of local sections of a signal as it changes over time.

At step 310, the match level is compared with a predetermined threshold (e.g., 90%). In response to determining that the match level is above the predetermined threshold, the process proceeds to step 312. In response to determining that the match level is below the predetermined threshold, the process proceeds to step 316.

The predetermined threshold may vary by patient. In addition, the predetermined threshold may be adjusted by the abnormality detection system 102 based on the status of the patient. For example, once an abnormality is detected the match level may be decreased to provide focused monitoring without requiring additional physicians. For example, after detecting an abnormality during a first respiratory cycle, the match level may be decreased by a predetermined value (e.g., 1%, 2%, or 5%). Thus, during a subsequent respiratory cycle a lower threshold is used. Further, in response to the detection engine 102 not detecting any abnormality in one or more subsequent respiratory cycles, the predetermined threshold may be increased by a predetermined incremental value (e.g., 1%). The predetermined threshold may be increased/decreased only for the category where the abnormality was detected or for all the categories. For example, if an abnormality is detected in a pressure scalar waveform the predetermined threshold when comparing shapes associated with a pressure scalar waveform is increased.

In one implementation, the detection engine 124 may request or acquire additional waveforms data or other physiological data from the patient physiological sensors 114 in response to determining that the match level is within a predetermined range ( e.g., between 50% and the predetermined threshold). By monitoring additional data on demand, processing speed is increased without compromising patient safety. For example, additional waveforms such as capnography can be analyzed when the match level for an abnormal shape associated with the PV loop is within the predetermined range.

At step 316, the match level may be stored in trend data 142 in data repository 134 for a predefined number of cycles (e.g., last 10 respiratory cycles). The match levels are monitored to determine a trend that may be indicative of a potential abnormality. The detection engine 124 may output an alert to the physician when the trend indicates that the match level is increasing through the predefined number of cycles even though the match level may be less than the predetermined threshold. By monitoring a trend of the match level, early detection of abnormalities is possible which increases patient safety.

At step 312, the ventilator settings engine 130 may determine new ventilator settings based on the identified abnormality. The ventilator settings engine 130 may also update the settings based on physiological features, such as cardiovascular circulation, respiratory mechanics, tissue and alveolar gas exchange, short-term neural control mechanisms acting on the cardiovascular and/of respiratory functions, or the like.

The updated ventilator settings may be variously used. In one implementation, the notifications and alerts engine 132 updated settings are sent to a device (e.g., electronic devices 122a, 122b of FIG. 1) associated with a physician. The updated settings are not directly applied to the mechanical ventilator 104. The physician is then free to use professional judgement as to whether the updated settings should be implemented. If so, the physician may use electronic device 122a, 122b to accept the updated ventilator settings. For example, the notification including the updated settings may include an associated “accept” button. Once the notification and alerts engine 132 receives the acknowledgement from the physician, the abnormality detection system 102 may output a signal updating the ventilator settings 116 of the mechanical ventilator 104 via the communication circuitry 112. Further, the physician 120 may input changes to the updated settings. The abnormality detection system 102 transmits the changes to the mechanical ventilator 104 to automatically update the ventilator settings 116. In one implementation, the physician may enter the desired settings using the user interface 106 of the mechanical ventilator 104.

In one implementation, when the updated ventilator settings are within a predetermined threshold from the previous settings, the abnormality detection system 102 may output a signal updating the ventilator settings 116. Further, the ventilator settings 116 may be automatically updated when the settings falls within a predefined range.

At step 314, the detection engine 124 may output an alert to the physicians 120 when an abnormality is detected. The alert may be visual, audible, and/or tactile.

The depicted order and labeled steps are indicative of one embodiment of the presented method 300. Other steps and methods may be conceived that are equivalent in function, logic, or effect of one or more steps or portions thereof, of the illustrated method 300. Additionally, the format and symbols employed are provided to explain the logical steps of the method 300 and are understood not to limit the scope of the method 300.

Although the flow charts show specific orders of executing functional logic blocks, the order of executing the block blocks may be changed relative to the order shown, as will be understood by one of ordinary skill in the art. Also, two or more blocks shown in succession may be executed concurrently or with partial concurrence. For example, steps 312 and 314 may be executed concurrently. A first alert may be output to the physician when an abnormality is detected. The first alert may be followed by a second alert that includes the updated ventilator settings.

The abnormality detection system 102 can also trigger a monitoring process at preset time intervals (e.g., every 20 minutes, 30 minutes, 1 hour). The monitoring process may monitor a plateau pressure which is the pressure at the end of the inhalation phase of the respiratory cycle. Conventionally, a physician or a respiratory therapist measure the plateau pressure by performing a manual maneuver (i.e., End-inspiratory pause or inspiratory hold) that holds the air flow for a short period of time (0.5-1 sec) at the end of the inhalation and measuring the plateau pressure. Similarly, total PEEP may be measured to quantify auto PEEP, [Intrinsic (auto) PEEP=Total (measured) PEEP−Extrinsic (set) PEEP] by performing a manual maneuver (i.e., End-expiratory pause or expiratory hold) that holds the air flow for a short period of time (0.5-1 sec) at the end of the exhalation 606. Additionally, auto PEEP can be predicted by the presence of End Expiratory Flow (EEF) which does not require a maneuver to be measured as well as it can be displayed continuously (breath by breath). Commonly, the inspiratory and expiratory maneuvers are performed at least every 4 hours in selected patients. The physician may not be able to detect a change in the plateau pressure or auto PEEP until the next maneuver is performed. During the interval period, the plateau pressure may increase drastically and the patient may be receiving injurious levels of plateau pressure thus develop lung injury. On the other hand, auto PEEP may develop in pulmonary and non-pulmonary patients and is believed to be a major cause for patient-ventilator asynchrony particularly missed triggers. Continuous monitoring of EEF and Frequent automatic measuring/monitoring of the plateau pressure and auto PEEP via performing the End-inspiratory/Expiratory pause maneuvers provide the advantage of an earlier notification of any changes in the plateau pressure or in the intrinsic PEEP without adding to the inconvenience neither to the patient nor to the clinician. An exemplary process to monitor the plateau pressure and auto PEEP is shown in FIG. 4.

In one implementation, the monitoring engine 126 may also determine a driving pressure as a function of the plateau pressure and the PEEP total. For example, the driving pressure may be expressed as: Driving pressure=plateau pressure−PEEP total. A target value for the driving pressure may be less than 15 cmH₂O. The monitoring engine 126 may output an alert in response to determining that the driving pressure is greater than the target value.

In one implementation, the monitoring engine 126 may determine a trans pulmonary pressure (Ptp) as a function of a alveolar pressure (P_{A}) and a pleural pressure (P_{L}). For example, the Ptp may be expressed as Ptp=P_{A}−P_{L}. A predetermined maximum threshold for the end-inspiratory Ptp (Ptpₚₗₐₜ) may be 20 or 25 cmH₂O. A higher Ptpₚₗₐₜ may indicate global lung stress and overdistention. An end-expiratory Ptp (Ptp_{PEEP}) may normally range between 0 to 10 cmH₂O. The monitoring engine 126 may output an alert in response to determining that the Ptp_{PEEP} falls outside the predetermined range. The Ptp_{PEEP} may also be used to determine the optimal PEEP level and to prevent atelectrauma that happens when Ptp_{PEEP} has a negative value.

In one implementation, the monitoring engine 126 may determine a lung stress value (i.e., equal to Ptpₚₗₐₜ) as a function of a constant K and strain. The constant may be equal to 13.5. The strain may be expressed as Strain=Vt/functional residual capacity (FRC). The lung stress may be expressed as Stress=K×strain. A predetermined maximum threshold for the lung stress may be 20. Thus, in response to the monitoring engine 126 determining that the lung stress is greater than 20, the monitoring engine 126 may output an alert to the physician 120.

The esophageal pressure measurement requires insertion of an esophageal catheter. The esophageal pressure may be used as a surrogate for the pleural pressure. The esophageal graph and value may be displayed based on the esophageal catheter pressure measurements. The esophageal graph can be used to identify ineffective patient triggering and diaphragmatic activity and serves as a tool to identify and quantify intrinsic PEEP. Monitoring the esophageal graph contributes to protective lung ventilation and patient-ventilator synchrony as well as in weaning success/failure prediction.

The driving pressure, the Ptp, and the lung stress are automatically measured and calculated at predetermined instances, on demand, or whenever a change is made in specific ventilator settings for monitoring and trending purposes. Monitoring the driving pressure, the Ptp, and the lung stress provides protective lung ventilation by continuously checking that these values are within the predefined range. When the values are out of the range associated with each of the driving pressure, the Ptp, and the lung stress or when an indication of ineffective triggering is detected, the abnormality detection engine 102 alerts the physicians because the out of ranges values are indicative of VILI and mortality.

FIG. 4 is a flowchart of a monitoring process 400 according to one example. At step 402, the test engine 128 may acquire end-inspiratory/expiratory hold test settings from data repository 134 associated with the mechanical ventilator 104. The end-inspiratory/expiratory hold test settings may include a preset time interval value, duration of each maneuver and frequency of maneuvers per attempt (e.g. 3 maneuvers, 3-5 breaths apart) to ensure the reliably of the obtained value.

At step 404, the test engine 128 may output to the mechanical ventilator 104 a signal to control the ventilator settings 116 based on the test settings. For example, a mechanical breath may be held at the end of inhalation or end of exhalation for 0.5 second or other value as set by the physician.

At step 406, the test engine 128 determines a plateau value and/or auto PEEP value from data received from the mechanical ventilator 104. For example, the plateau pressure and auto PEEP values may be determined from the pressure scalar waveform. Exemplary pressure scalar waveforms are shown in FIGS. 6A-6C.

At step 408, the test engine 128 checks to see whether the plateau pressure and/or auto PEEP values determined at step 406 are within the predefined range. At step 410, in response to determining that the obtained value is not within the predefined range, an alert is output to the physician associated with the mechanical ventilator 104. In response to determining that the plateau pressure and/or auto PEEP value is increasing or decreasing, but within the predetermined range, an alert is output to the physician associated with the mechanical ventilator 104. In response to determining that the plateau pressure and/or auto PEEP value is unchanged (±1 cmH₂O), and within the predetermined range, the process proceeds to step 402 where the process may be repeated at the preset time intervals. In addition, the plateau pressure and/or auto PEEP value is stored/updated in their designated database (i.e., plateau pressure data 140 and Auto PEEP data 144). The plateau pressure data is stored in 140 and the auto PEEP data is stored in 144.

Conventionally, ventilators have predetermined thresholds for volume/pressure that when reached trigger an alarm for low volume/pressure which may indicate a leak during mechanical ventilation (e.g., due to a fault in the mechanical ventilator, breathing circuit, endotracheal tube 104 or an abnormality). The abnormality detection system 102 described herein may monitor the FV loop data 208 Mid the volume scalar data 204 to determine whether there is as indication of a leak. Thus, the leak is detected before reaching the low volume/pressure conventional limits, which allows early notification to the physicians 120 and the controller 110 to correct or modify settings before irreversible damages may occur to the ventilated patient 118.

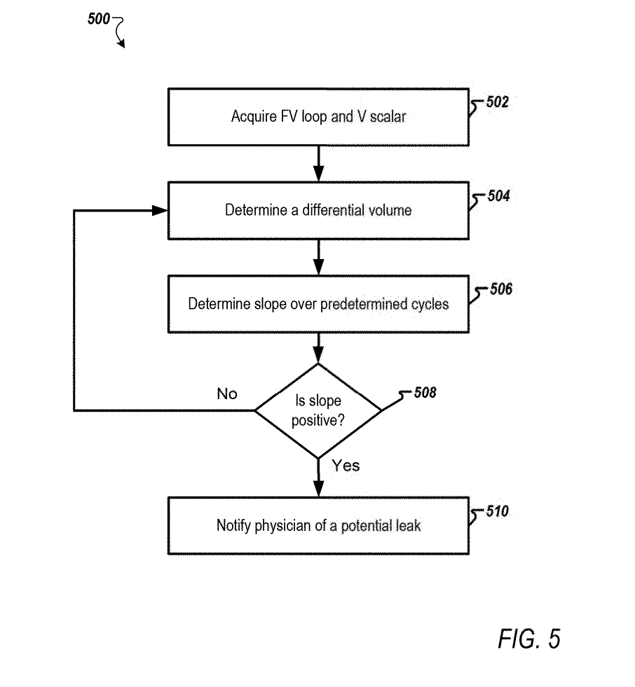

FIG. 5 is a flowchart of a leak detection process 500 according to one example.

At step 502, the monitoring engine 126 may acquire datasets associated with FV loop 208 and volume scalar 204. At step 504, the monitoring engine 126 may determine a differential volume (i.e., a difference between inspiration volume and expiration volume). Then, the differential volume may be stored in the trend data 142.

At step 506, the monitoring engine 126 may determine a slope associated with differential volumes acquired over a predefined number of cycles (e.g., determine the slope of the differential volume for the last predefined number of cycles).

At step 508, the monitoring engine 126 may check to see whether the slope is positive. In response to determining that the slope is positive, the process proceeds to step 510. In response to determining that the slope is not positive, the process goes back to step 504. Monitoring a slope or an increasing trend over the predefined number of cycles has the advantage of differentiating between small leaks and normal fluctuations due to noise from various elements of the medical ventilation system 100, thus providing early detection of leaks while minimizing the possibility of false alarms.

At step 510, an alert is output to the electronic device 122 associated with a physician monitoring (i.e., supervising) the mechanical ventilator 104. The alert may include a representation of the differential volumes over the predefined number of cycles.

FIGS. 6A-6L are schematics that show exemplary abnormal waveforms according to one example. The exemplary abnormal waveforms may be stored as the abnormal shapes data 136 in the data repository 134.

FIGS. 6A-6C show abnormal pressure scalar waveforms. Graph 602 shows a pressure scalar waveform, where the difference between the PIP and Pplat is high (ΔP_{PIP-Pplat}). The difference between PIP and Pplat represents airway resistance. Pplat represents lung and/or chest wall compliance. In graph 602, the Pplat is high. The abnormality detection system 102 may compare the waveform shown in graph 602 and a waveform acquired from the ventilator 104 to determine the match level. In response to detecting a match between the waveform of graph 602 and the waveform, the abnormality detection system 102 may output a notification including possible intervention steps to decrease the airway resistance, such as administrating drugs, clearing the airway, or changing the tube. Graph 604 shows a pressure scalar waveform with a decreased lung/and or chest wall compliance abnormality. Graph 606 shows a pressure scalar waveform that indicates an air trapping abnormality. In graphs 602 and 604, there is an inspiratory “pause”. In graph 606, there is an expiratory “pause”. Graph 608 shows a pressure scalar waveform that shows a morphology indicating a pressure overshoot abnormality (i.e., the rise time is too fast). The pressure overshoot abnormality may be detected in a pressure scalar waveform acquired from the ventilator 104 by monitoring the slope of the pressure. Graph 610 shows a pressure scalar waveform that indicates a flow starvation abnormality. A first breath reveals inadequate respiration flow rate leading to asynchrony manifested by scooped-out pressure waveform. Graph 612 shows a pressure scalar waveform that shows missed triggers. In one implementation, once a missed triggers abnormality is detected, the abnormality detection system 102 may modify the ventilator settings. For example, a higher sensitivity setting may be used. Graph 614 shows a pressure scalar waveform that is indicative of an active exhalation abnormality. The active exhalation abnormality may be detected by monitoring the inspiratory time. A long inspiratory time indicates the active exhalation abnormality. Graphs 616, 618, and 620 show segments of pressure scalar waveforms that may be used to monitor a stress index. Graph 616 shows a convex curve which may indicate recruitment. Graph 618 shows a linear curve which may indicate that there is no recruitment or overdistention. Graph 620 shows a concave curve which may indicate overdistention. Thus, the abnormality detection system 102 may determine whether a predetermined segment of the pressure scalar waveform is nonlinear which may indicate overdistention or recruitment. Further, the abnormality detection system 102 may determine whether the segment is convex or concave to determine whether the abnormality is recruitment or overdistention.

FIGS. 6D and 6E are schematics that show exemplary abnormal volume scalar waveforms. Graph 622 shows an abnormal scalar waveform that indicates an air-trapping or leak. The delivered tidal volume has not fully returned to the ventilator 104. Graph 624 shows an abnormal scalar waveform that may indicate an air leak. As shown in graph 624, the volume does not return to the baseline. Graph 626 shows an abnormal volume scalar waveform that is indicative of Biot (i.e., cluster) respiration. Graph 626 shows clustering of rapid and shallow breaths coupled with regular or irregular periods of apnea. Graph 628 shows an abnormal volume scalar waveform that is indicative of Cheyne-Stockes respiration. Breaths gradually increase and decrease in depth and rate with periods of apnea. Thus, in response so the abnormality detection system 102 detecting that the rate and depths of the breaths are irregular, the abnormality detection system 102 may detect the Cheyne-Stockes abnormality. Graph 630 shows an abnormal volume scalar waveform that is indicative of Kussmaul breathing ( i.e., deep and fast respirations). Graph 632 shows an abnormal volume scalar waveform that is indicative of apneustic breathing (i.e., deep, gasping inspiration with brief partial expiration). Graph 634 shows an abnormal volume scalar waveform that is indicative of ataxic breathing. The graph 634 shows completely irregular breathing pattern with variable periods of apnea.

FIGS. 6F and 6G are schematics that show abnormal flow scalar waveforms. Graph 636 shows an abnormal flow scalar waveform that is indicative of air-trapping. There is remaining air flow at the end of exhalation. In normal flow scalar waveform the expiratory flow returns to zero (i.e., baseline) before the start of next breath. Graph 638 is an abnormal flow scalar waveform that is indicative of missed triggers. Missed triggers may be caused by low sensitivity settings. The abnormality detection system 102 may adjust the settings to increase the sensitivity level. Graph 640 shows an abnormal flow scalar waveform that is indicative of auto triggering. Breath triggers by air leak that increases respiratory rate and may falsely considered as tachypnea. Graph 642 shows an abnormal flow scalar waveform that is indicative of double triggering. Double triggering occurs when the patient inspiratory demand has not been fully fulfilled. Schematic 644 shows exemplary abnormal flow scalar waveforms that are indicative of ineffective triggers. The ineffective triggers in schematic 644 appeared in the inspiratory and expiratory phases, but can occur on either phase. Graph 646 shows an exemplary waveform that shows the response to therapy and/or interventions. Therapies can include medications, aerosol, or airway clearance. Graph 648 shows an abnormal flow scalar waveform that has a sawtooth appearance that may indicate the presence of secretion or rain out in the expiratory circuit.

FIGS. 6H and 6I are schematics that show abnormal PV loops according to one example. Graph 650 may be used to identify LIP and UIP. Ventilating patients above LIP (to prevent atelectrauna) and below UIP (to prevent overdistention) known as “Open Lung Ventilation” is believed to be the safest ventilation zone that achieves “Protective Lung Strategy”. So optimal levels of PEEP and maximum pressure (Pisp) may be identified. Graph 652 shows an abnormal PV loop that is indicative of overdistentetion. Graph 654 shows an abnormal PV loop that is indicative of increased airway resistance on inspiratory and expiratory. Graph 656 shows an abnormal PV loop that is indicative of a leak. Graph 658 shows an abnormal PV loop that is indicative of increased expiratory resistance. Graph 660 shows an abnormal PV loop that is indicative of increased inspiratory resistance.

FIGS. 6J and 6K are schematic that show abnormal FV loops. Graph 662 shows an abnormal FV loop that is indicative of a leak. Graph 664 shows an abnormal FV loop that is indicative of increased expiratory resistance. Graph 666 shows an abnormal FV loop that is indicative of increased inspiratory resistance. Graph 668 shows an abnormal FV loop that is indicative of air trapping. Graph 670 shows an abnormal FV loop that is indicative of active exhalation.

FIG. 6L is a schematic that shows abnormal capnography waveforms. Graph 672 is an abnormal capnography that is indicative of CO₂ rebreathing. Graph 674 is an abnormal capnography that is indicative of hypoventilation. As shown in graph 674, the CO₂ level is increasing. Graph 676 is an abnormal capnography that is indicative of hyperventilation. As shown in graph 676, the CO₂ level is decreasing. Graph 678 shows an abnormal capnography that is indicative of partial airway obstruction. Partial airway obstruction may occur due to secretion accumulation in the airway or bronchoconstriction. Graph 680 shows an abnormal capnography that is indicative of air leak. This may be due to inadequate cuff pressure. Next, a hardware description of a computer 728 according to exemplary embodiments is described with reference to FIG. 7. In FIG. 7, the computer 728 includes a CPU 700 which performs the processes described herein. The process data and instructions may be stored in memory 702. These processes and instructions may also be stored on a storage medium disk 704 such as a hand drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computer 728 communicates, such as a server or computer.

Further, the claimed advancements may be provided as a utility application, background daemon, or component of an operating system, or combination thereof, executing in conjunction with CPU 700 and an operating system such as Microsoft® Windows®, UNIX®, Oracle® Solaris, LINUX®, Apple macOS™ and other systems known to those skilled in the art.

In order to achieve the computer 728, the hardware elements may be realized by various circuitry elements, known to those skilled in the art. For example, CPU 700 may be a Xenon or Core processor from Intel of America or an Opteron processor from AMD of America, or may be other processor types that would be recognized by one of ordinary skill in the art. Alternatively, the CPU 700 may be implemented on an FPGA, ASIC, PLD or using discrete logic circuits, as one of ordinary skill in the art would recognize. Further, CPU 700 may be implemented as multiple processors cooperatively working in parallel to perform the instructions of the inventive processes described above.

The computer 728 in FIG. 7 also includes a network controller 706, such as an Intel Ethernet PRO network interface card from Intel Corporation of America, for interfacing with network 730. As can be appreciated, the network 730 can be a public network, such as the Internet, or a private network such as LAN or WAN network, or any combination thereof and can also include PSTN or ISDN sub-networks. The network 730 can also be wired, such as an Ethernet network, or can be wireless such as a cellular network including EDGE, 3G and 4G wireless cellular systems. The wireless network can also be WiFi®, Bluetooth®, or any other wireless form of communication that is known that is pre-registered, verified and highly secured.

The computer 728 further includes a display controller 708, such as a, NVIDIA® GeForce® GTX or Quadro® graphics adaptor from NVIDIA Corporation of America for interfacing with display 710, such as a Hewlett Packard® HPL2445w LCD monitor. A general purpose I/O interface 712 interfaces with a keyboard and/or mouse 714 as well as an optional touch screen panel 716 on or separate from display 710. General purpose I/O interface also connects to a variety of peripherals 718 including printers and scanners, such as an OfficeJet® or DeskJet® from Hewlett Packard.

A sound controller 720 is also provided in the computer 728, such as Sound Blaster® X-Fi Titanium® from. Creative, to interface with speakers/microphone 722 thereby providing sounds and/or music.

The general purpose storage controller 724 connects the storage medium disk 704 with communication bus 726, which may be an ISA, EISA, VESA, PCI, or similar, for interconnecting all of the components of the computer 728. A description of the general features and functionality of the display 710, keyboard and/or mouse 714, as well as the display controller 708, storage controller 724, network controller 706, sound controller 720, and general purpose I/O interface 712 is omitted herein for brevity as these features are known.

The exemplary circuit elements described in the context of the present disclosure may be replaced with other elements and structured differently than the examples provided herein.

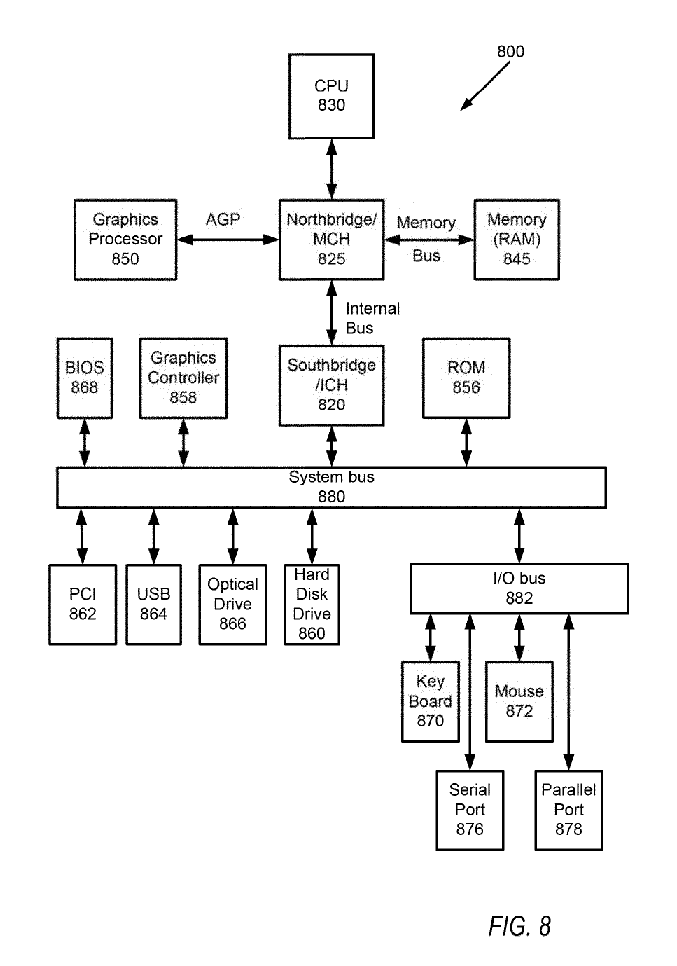

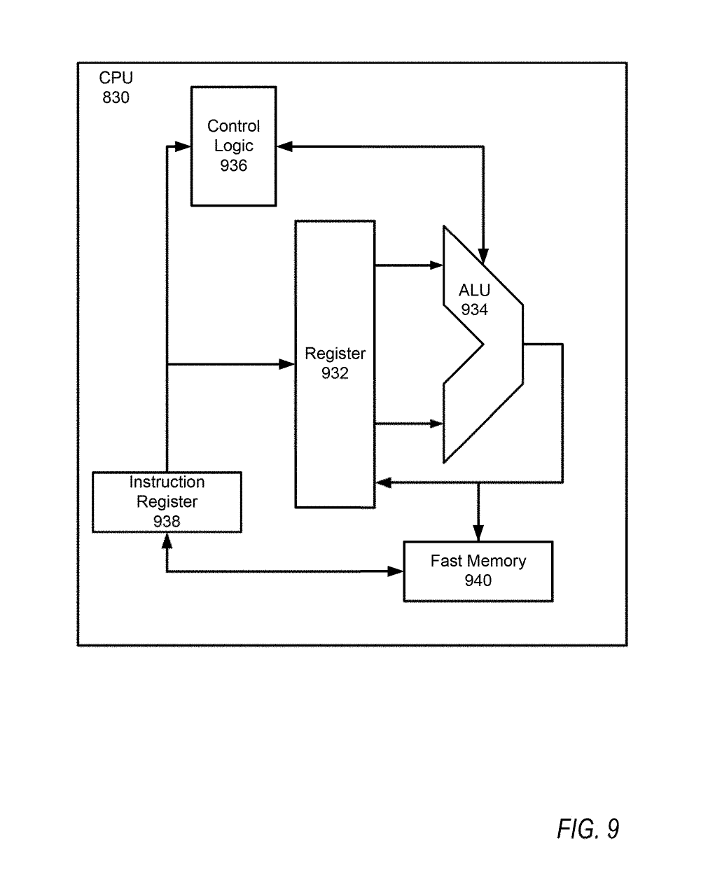

FIG. 8 shows a schematic diagram of a data processing system, according to certain embodiments, for analyzing and monitoring respiratory waveforms utilizing the methodologies described herein. The data processing system is an example of a computer in which specific code or instructions implementing the processes of the illustrative embodiments may be located to create a particular machine for implementing the above-noted process.

In FIG. 8, data processing system 800 employs a hub architecture including a north bridge and memory controller hub (NB/MCH) 825 and a south bridge and input/output (I/O) controller hub (SB/ICH) 820. The central processing unit (CPU) 830 is connected to NB MCH 825. The NB/MCH 825 also connects to the memory 845 via a memory bus, and connects to the graphics processor 850 via an accelerated graphics port (AGP). The NB/MCH 825 also connects to the SB/ICH 820 via an internal bus (e.g., a unified media interface or a direct media interface). The CPU 830 may contain one or more processors and may even be implemented using one or more heterogeneous processor systems. For example, FIG. W shows one-implementation of CPU 830.

Further, in the data processing system 800 of FIG. 8, SB/ICH 820 is coupled through a system bus 880 to an I/O Bus 882, a read only memory (ROM) 856, an universal serial bus (USB) port 864, a flash binary input/output system (BIOS) 868, and a graphics controller 858. In one implementation, the I/O bus can include a super I/O (SIO) device.

PCI/PCIe devices can also be coupled to SB/ICH 820 through a PCI bus 862. The PCI devices may include, for example, Ethernet adapters, add-in cards, and PC cards for notebook computers. Further, the hard disk drive (HDD) 860 and optical drive 866 can also be coupled to the SB/ICH 820 through the system bus 880. The Hard disk drive 860 and the optical drive or CD-ROM 866 can use, for example, an integrated drive electronics (IDE) or serial advanced technology attachment (SATA) interface.

In one implementation, a keyboard 870, a mouse 872, a serial port 876, aid a parallel port 878 can be connected to the system bus 880 through the I/O bus 882. Other peripherals and devices that can be connected to the SB/ICH 820 include a mass storage controller such as SATA or PATA (Parallel Advanced Technology Attachment), an Ethernet port, an ISA bus, a LPC bridge, SMBus, a DMA controller, and an Audio Codec (not shown).

In one implementation of CPU 830, the instruction register 938 retrieves instructions from the fast memory 940. At least part of these instructions are fetched from the instruction register 938 by the control logic 936 and interpreted according to the instruction set architecture of the CPU 930. Part of the instructions can also be directed to the register 932. In one implementation, the instructions are decoded according to a hardwired method, and in another implementation, the instructions are decoded according a microprogram that translates instructions into sets of CPU configuration signals that are applied sequentially over multiple clock pulses. After fetching and decoding the instructions, the instructions are executed using the arithmetic logic unit (ALU) 934 that loads values from the register 932 and performs logical and mathematical operations on the loaded values according to the instructions. The results from these operations can be feedback into the register and/or stored in the fast memory 940. According to certain implementations, the instruction set architecture of the CPU 830 can use a reduced instruction set architecture, a complex instruction set architecture, a vector processor architecture, a very large instruction word architecture. Furthermore, the CPU 830 can be based on the Von Neuman model or the Harvard model. The CPU 830 can be a digital signal processor, an FPGA, an ASIC, a PLA, a PLD, or a CPLD. Further, the CPU 830 can be an x86 processor by Intel or by AMD; an ARM processor, a Power architecture processor by, e.g., IBM; a SPARC architecture processor by Sun Microsystems or by Oracle; or other known CPU architecture.

The present disclosure is not limited to the specific circuit elements described herein, nor is the present disclosure limited to the specific sizing and classification of these elements.

The functions and features described herein may also be executed by various distributed components of a system. For example, one or more processors may execute these system functions, wherein the processors are distributed across multiple components communicating in a network. The distributed components may include one or more client and server machines, which may share processing in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely from ventilators either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

The hardware description above, exemplified by any one of the structure examples shown in FIGS. 7 or 8, constitutes or includes specialized corresponding structure that is programmed or configured to perform the algorithm shown in FIGS. 3,4, or 5.

A system which includes the features in the foregoing description provides numerous advantages to users. In particular, the system and associated methodologies provides early detection of abnormalities that could have been overlooked or discovered late while using a mechanical ventilator. In addition, the system automatically generates alerts to physicians when an indication of an abnormality is detected that lead to early prevention of complications associated with the mechanical ventilators. The system described herein promotes patient-ventilator synchrony. Advancement in processing and computing technologies provides the ability to manipulate and process waveforms data according to the implementations described herein. The methodologies described herein could not be implemented by a human due to the sheer complexity of waveform analyzing in real time that results in significantly more than any construed abstract idea. The system and associated methodologies described herein provide a technical solution to the technical problem of optimally controlling a ventilator and detecting abnormalities.

Obviously, numerous modifications and variations are possible in light of the above teachings. It is therefore to be understood that within the scope of the appended claims, the invention may be practiced otherwise than as specifically described herein.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A method for monitoring respiratory waveforms, the method comprising:
acquiring a data set representative of a waveform;
comparing, using processing circuitry, one or more segments of the data set with stored abnormal shapes and/or values;
determining, using the processing circuitry and based on the comparison, a match level;
identifying an abnormality associated with an abnormal shape and/or a value in response to determining that the match level between the data set and the abnormal shape and/or the value is above greater or below a predetermined threshold; and
outputting a notification indicating the abnormality to an external device.

2. The method of claim 1, wherein the step of comparing includes:
segmenting the data set into multiple segments associated with phases of a respiratory cycle of a patient.

3. The method of claim 1, wherein the step of comparing includes:
determining a first derivative of the one or more segments of the data sets; and
comparing the first derivative of the one or more segments with first derivatives of abnormal shapes and/or values.

4. The method of claim 1, further comprising:
storing the match level associated with a waveform category;
identifying a trend based on stored match levels; and
outputting an alert when the trend is indicative of a potential abnormality.

5. The method of claim 4, wherein an increase in the match level over a predetermined number of successive data sets is indicative of the potential abnormality.

6. The method of claim 1, wherein the predetermined threshold for subsequent comparisons is decreased when an abnormality is detected.

7. The method of claim 1, further comprising;
acquiring a second data set representative of a second waveform of a different category when the match level is within a predetermined range.

8. The method of claim 1, wherein the waveform includes a pressure scalar, a volume scalar, a flow scalar, a flow volume loop, or a pressure volume loop.

9. The method, of claim 1, wherein the data set is acquired from a mechanical ventilator.

10. The method of claim 9, further comprising:
determining updated ventilator settings in response to determining that the match level is above greater or below a predetermined threshold;
outputting the updated ventilator settings to the external device;
acquiring an input from the external device; and
controlling settings of the mechanical ventilator based on the physician input and the updated ventilator settings.

11. The method of claim 9, further comprising:
controlling one or more parameters of the mechanical ventilator at preset time intervals;
acquiring data from the mechanical ventilator;
determining a plateau pressure, an auto positive end-expiratory pressure (PEEP), driving pressure, an end inspiratory pressure (Ptpₚₗₐₜ), an end expiratory pressure (Ptpₚₑₑₚ), and a pressure difference between a peak inspiratory pressure and the plateau pressure (ΔP_{PIP-Pplat}); and
alerting the physician in response to determining that the plateau pressure, the auto PEEP, driving pressure, Ptpₚₗₐₜ, Ptpₚₑₑₚ, or ΔP_{PIP-Pplat} are not within a predetermined pressure range.

12. The method of claim 11, wherein controlling the one or more parameters include holding the mechanical breath for 0.5 seconds.

13. The method of claim 1, further comprising:
acquiring one or more data sets associated with volume scalar data;
determining a differential volume based on volume scalar data;
determining a slope associated with differential volumes determined for successive respiratory cycles;
identifying a leak in response to determining that the slope is positive; and
outputting an alert to the external device in response to identifying a leak.

14. The method of claim 1, further comprising:
maintaining a predetermined cuff pressure by monitoring data from a monometer.

15. The method of claim 1, further comprising:
acquiring a measure of exhaled nitric oxide;
monitoring the measure of exhaled nitric oxide; and
identifying a trend based on the monitoring.

16. The method of claim 1, further comprising:
acquiring a humidity level of inspired air via a humidity sensor;
determining whether the humidity level is within a predetermined humidification range; and
outputting the notification indicating an abnormality in the humidity level to the external device when the humidity level in not within the predetermined humidification range.

17. A mechanical ventilator system, the system comprising:
a mechanical ventilator; and
processing circuitry configured to
acquire a data set representative of a waveform from the mechanical ventilator,
compare one or more segments of the data set with stored abnormal shapes and/or values,
determine a match level based on the comparison,
identify an abnormality associated with an abnormal shape and/or a value in response to determining that the match level between the data set and the abnormal shape and/or value is above greater or below a predetermined threshold, and
output a notification indicating the abnormality to an external device.

18. The system of claim 17, wherein the processing circuitry is further configured to:
segment the data set into multiple segments associated with phases of a respiratory cycle of a patient.

19. The system of claim 17, wherein the processing circuitry is further configure to:
determine a first derivative of the one or more segments of the data sets; and
compare the first derivative of the one or more segments with first derivatives of abnormal shapes and/or values.

20. A non-transitory computer readable medium storing computer-readable instructions therein which when executed by a computer cause the computer to perform a method for monitoring respiratory waveforms, the method comprising:
acquiring a data set representative of a waveform;
comparing one or more segments of the data set with stored abnormal shapes and/or values;
determining a match level based on the comparison;
identifying an abnormality associated with an abnormal shape and/or value to response to determining that the match level between the data set and the abnormal shape and/or value is above greater or below a predetermined threshold; and
outputting a notification indicating the abnormality to an external device.
